(19) United States
(12) Patent Application Publication
(10) Pub. No.: US 2022/0188601 A1
(43) Pub. Date: Jun. 16, 2022

(54) SYSTEM IMPLEMENTING ENCODER-DECODER NEURAL NETWORK ADAPTED TO PREDICTION IN BEHAVIORAL AND/OR PHYSIOLOGICAL CONTEXTS

(71) Applicant: Cornell University, Ithaca, NY (US)

(72) Inventors: Daniel A. Adler, Yardley, PA (US); Tanzeem Choudhury, Pittsford, NY (US); Vincent W.-S. Tseng, New York, NY (US)

(21) Appl. No.: 17/551,994

(22) Filed: Dec. 15, 2021

Related U.S. Application Data
(60) Provisional application No. 63/125,677, filed on Dec. 15, 2020.

(51) Int. Cl. G06N 3/04

(52) U.S. Cl. CPC G06N 3/0454; G06N 3/0445

(57) ABSTRACT

A method in an illustrative embodiment comprises obtaining data characterizing a given subject over time, applying at least a portion of the obtained data to an encoder-decoder neural network adapted to generate a prediction of at least one change in at least one of behavior and physiology of the given subject from the obtained data, and executing at least one automated remedial action relating to the given subject based at least in part on the generated prediction. The encoder-decoder neural network is configured to learn one or more subject-specific anomaly thresholds based at least in part on reconstruction error of the encoder-decoder neural network. The encoder-decoder neural network is illustratively implemented utilizing at least one of a fully-connected neural network autoencoder architecture and a gated recurrent unit sequence-to-sequence architecture. Other illustrative embodiments include systems and computer program products.

### RELATED APPLICATION

The present application claims priority to U.S. Provisional Patent Application Ser. No. 63/125,677, filed Dec. 15, 2020, which is incorporated by reference herein in its entirety.

### FIELD

The field relates generally to information processing systems, and more particularly to machine learning and other types of artificial intelligence implemented in such systems.

### BACKGROUND

Behavioral and/or physiological analysis is fundamental in numerous information processing contexts, including diverse fields such as healthcare, security and sports. Conventional approaches to behavioral and/or physiological analysis are problematic in that such approaches often require extensive manual intervention by highly trained personnel, and can therefore lead to excessive costs and other difficulties in analyzing both simple and complex behaviors and/or physiologies in a repeatable and scalable manner. Moreover, conventional approaches that attempt to apply automation in these contexts fail to adequately detect behavioral and/or physiological changes associated with increased disease risk.

### SUMMARY

Illustrative embodiments provide systems implementing encoder-decoder neural networks (EDNNs) adapted to prediction in behavioral contexts, physiological contexts, and/or in numerous other contexts. For example, some embodiments provide a system adapting one or more EDNNs to predict early warning signs of various behavioral and/or physiological conditions. In some embodiments, this more particularly involves predicting behavioral and/or physiological changes that are indicative of certain behavioral and/or physiological conditions, illustratively using passive sensing data collected, with little to no user interaction, from one or more mobile sensors (e.g., a smartphone and/or one or more wearable devices). Behavioral anomalies are often an early warning sign of mental health deterioration across a variety of conditions, including depression and psychosis. Accordingly, some embodiments disclosed herein predict early warning signs of psychotic relapse from passive sensing data. Other embodiments are applied in a wide variety of other use cases.

One or more such embodiments illustratively further provide various types of automated remediation responsive to predictions generated by the one or more EDNNs. For example, some embodiments implement EDNN-based prediction and remediation algorithms to at least partially automate various aspects of patient care in healthcare applications such as telemedicine. Such applications can involve a wide variety of different types of remote medical monitoring and intervention.

In one embodiment, a method comprises obtaining data characterizing a given subject over time, applying at least a portion of the obtained data to an EDNN adapted to generate a prediction of at least one change in at least one of behavior and physiology of the given subject from the obtained data, and executing at least one automated remedial action relating to the given subject based at least in part on the generated prediction. The EDNN is configured to learn one or more subject-specific anomaly thresholds based at least in part on reconstruction error of the EDNN. The EDNN is illustratively implemented utilizing at least one of a fully-connected neural network autoencoder architecture and a gated recurrent unit (GRU) sequence-to-sequence architecture. Other types of EDNN architectures and associated models and algorithms can be used in other embodiments.

In some embodiments, generating a prediction of at least one change in at least one of behavior and physiology of the given subject from the obtained data comprises detecting an anomaly based at least in part on reconstructed data generated by the EDNN from corresponding input data.

For example, detecting the anomaly in some embodiments comprises computing reconstruction error between the reconstructed data and the input data, comparing the reconstruction error to a particular one of the one or more subject-specific anomaly thresholds, and detecting the anomaly responsive to the reconstruction error exceeding the subject-specific anomaly threshold.

In some embodiments, executing at least one automated remedial action relating to the subject based at least in part on the generated prediction illustratively comprises generating at least one output signal in a telemedicine application. For example, such output signals in a telemedicine application can comprise a prediction visualization signal for presentation on a user terminal, diagnosis information transmitted over a network to a medical professional, and/or prescription information transmitted over a network to a prescription-filling entity. A wide variety of other signals can be generated in conjunction with execution of one or more automated remedial actions in illustrative embodiments. For example, one or more prediction-driven control signals can be integrated into behavioral and/or wearable technologies for self-intervention. This illustratively includes utilizing control signals generated in the manner disclosed herein to provide a user with recommendations for behavioral interventions via a smartphone, wearable or other type of user device.

It is to be appreciated that the foregoing arrangements are only examples, and numerous alternative arrangements are possible.

These and other illustrative embodiments include but are not limited to systems, methods, apparatus, processing devices, integrated circuits, and computer program products comprising processor-readable storage media having software program code embodied therein.

### BRIEF DESCRIPTION OF THE FIGURES

FIG. 1 is a block diagram of an information processing system comprising a processing platform implementing an EDNN adapted to prediction in contexts such as behavior and physiology in an illustrative embodiment.

FIG. 2 shows an example of an EDNN architecture for a fully-connected autoencoder model in an illustrative embodiment.

FIG. 3 shows an example of an EDNN architecture for a GRU sequence-to-sequence model in an illustrative embodiment.

FIG. 4 shows overall results of experiments performed using the example EDNN architectures of FIGS. 2 and 3.

FIG. 5 shows example behavioral changes for individual participants in the experiments to predict behavioral anomalies associated with relapse in schizophrenia using the example EDNN architectures of FIGS. 2 and 3.

FIG. 6 shows an example of an anomaly detection system configured to predict relapse in schizophrenia and to provide clinical intervention in response thereto in an illustrative embodiment.

FIG. 7 is a flow diagram of an example process utilizing an EDNN adapted to prediction in contexts such as behavior and physiology in an illustrative embodiment.

### DETAILED DESCRIPTION

Illustrative embodiments can be implemented, for example, in the form of information processing systems comprising one or more processing platforms each having at least one computer, server or other processing device. A number of examples of such systems will be described in detail herein. It should be understood, however, that embodiments of the invention are more generally applicable to a wide variety of other types of information processing systems and associated computers, servers or other processing devices or other components. Accordingly, the term “information processing system” as used herein is intended to be broadly construed so as to encompass these and other arrangements.

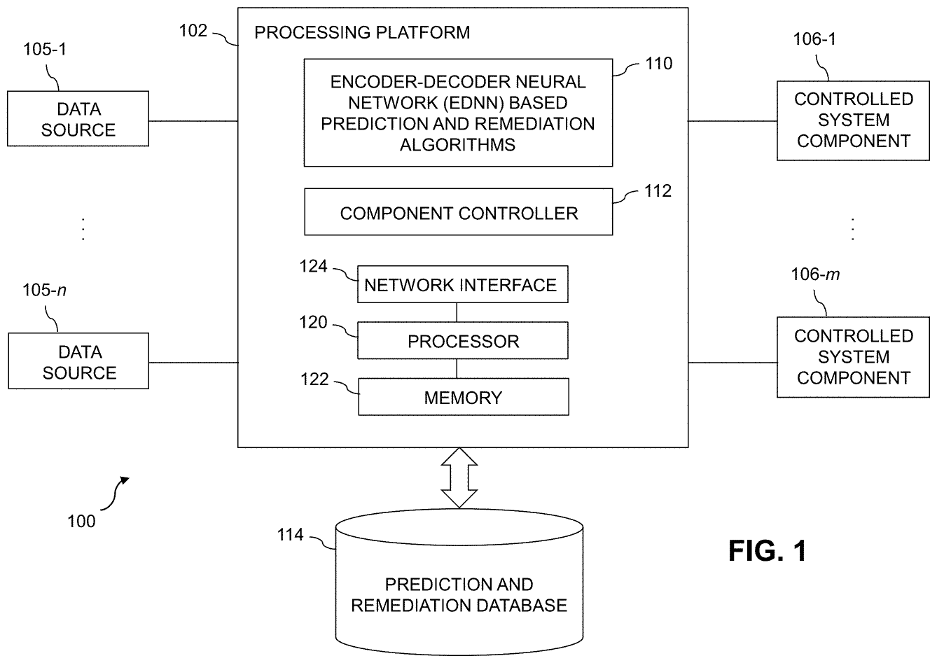

FIG. 1 shows an information processing system 100 implementing an EDNN adapted to prediction in contexts such as behavior and/or physiology in an illustrative embodiment. The system 100 comprises a processing platform 102. Coupled to the processing platform 102 are data sources 105-1, . . . 105-n and controlled system components 106-1, . . . 106-m, where n and m are arbitrary integers greater than or equal to two and may but need not be equal. Other embodiments can include only a single data source and/or only a single controlled system component. The processing platform 102 implements one or more EDNN-based algorithms 110 and at least one component controller 112. The EDNN-based algorithms 110 in the present embodiment more particularly comprise EDNN-based prediction and remediation algorithms, although other arrangements are possible.

In operation, the processing platform 102 is illustratively configured to obtain, from one or more of the data sources 105, data characterizing a given subject over time, to apply at least a portion of the obtained data to at least one EDNN implemented in the EDNN-based algorithms 110 to generate a prediction of at least one change in at least one of behavior and physiology of the given subject from the obtained data, and to execute at least one automated remedial action relating to the given subject based at least in part on the generated prediction, illustratively via the component controller 112.

For example, the data may be obtained from at least one of one or more wearable devices of the given subject, a smartphone of the given subject, and one or more sensors associated with the given subject. The generated prediction can comprise, for example, an indicator of a predicted health condition relapse or other particular predicted behavioral and/or physiological condition of the given subject, although a wide variety of other types of predictions can be generated using the EDNN-based algorithms 110 in other embodiments.

A given EDNN implemented in processing platform 102 is illustratively configured to learn one or more subject-specific anomaly thresholds based at least in part on reconstruction error of the EDNN. Detailed examples of such subject-specific anomaly thresholds are provided elsewhere herein. In some embodiments, the subject-specific anomaly thresholds more particularly provide patient-specific relapse signatures for respective ones of a plurality of patients with schizophrenia spectrum disorders (SSDs).

The learning of the EDNN is illustratively performed across multiple distinct features characterizing subject behavior and/or physiology. For example, in some embodiments, the multiple distinct features comprise, for example, one or more of at least one social behavior measure, at least one sleep measure and at least one activity measure, as described elsewhere herein. Examples of additional measures include a heart rate measure, a mood measure, etc.

Also, it is to be appreciated that the term “feature” as used herein is intended to be broadly construed, and should not be viewed as being limited in any way to the particular features mentioned above or elsewhere herein. For example, in some embodiments, features can comprise respective multiple distinct data types.

The generated prediction illustratively comprises an indicator of the likely presence of one or more behavioral and/or physiological anomalies, or other specified conditions, such as a predicted relapse relating to a designated SSD. The term “prediction” as used herein is therefore intended to be broadly construed, and may indicate, for example, likely presence or absence of an anomaly or set of anomalies.

In some embodiments, generating a prediction of at least one change in at least one of behavior and physiology of the given subject from the obtained data comprises detecting an anomaly based at least in part on reconstructed data generated by the EDNN from corresponding input data.

For example, detecting the anomaly in some embodiments comprises computing reconstruction error between the reconstructed data and the input data, comparing the reconstruction error to a particular one of the one or more subject-specific anomaly thresholds, and detecting the anomaly responsive to the reconstruction error exceeding the subject-specific anomaly threshold.

In some embodiments, the generated prediction is illustratively associated with one or more predicted changes in mental health of the given subject, so as to permit interpretation of the generated prediction in the context of the mental health of the given subject.

Numerous other arrangements of multiple distinct features and associated generated predictions are possible. For example, as indicated above, one or more features can comprise multiple distinct data types in some embodiments.

It is to be appreciated that the term “EDNN-based algorithm” as used herein is intended to be broadly construed to encompass a prediction algorithm and/or a remediation algorithm operating at least in part utilizing an EDNN. Detailed examples of particular implementations of EDNN-based algorithms 110 in illustrative embodiments are described elsewhere herein. A given such algorithm can implement one or more EDNN models and other associated functionality of the type disclosed herein.

The component controller 112 generates one or more control signals for adjusting, triggering or otherwise controlling various operating parameters associated with the controlled system components 106 based at least in part on predictions generated by the EDNN-based algorithms 110. A wide variety of different types of devices or other components can be controlled by component controller 112, possibly by applying control signals or other signals or information thereto, including additional or alternative components that are part of the same processing device or set of processing devices that implement the processing platform 102. Such control signals, and additionally or alternatively other types of signals and/or information, can be communicated over one or more networks to other processing devices, such as user terminals associated with respective system users.

The processing platform 102 is configured to utilize a prediction and remediation database 114. Such a database illustratively stores user data, user profiles and a wide variety of other types of information, including data from one or more of the data sources 105, that may be utilized by the EDNN-based algorithms 110 in performing prediction and remediation operations. The prediction and remediation database 114 is also configured to store related information, including various processing results, such as predictions or other outputs generated by the EDNN-based algorithms 110.

The component controller 112 utilizes outputs generated by the EDNN-based algorithms 110 to control one or more of the controlled system components 106. The controlled system components 106 in some embodiments therefore comprise system components that are driven at least in part by outputs generated by the EDNN-based algorithms 110. For example, a controlled component can comprise a processing device such as a computer, a smartphone or a wearable device that presents a display to a user and/or directs a user to adjust its behavior in a particular manner responsive to an output of an EDNN-based algorithm.

These and numerous other different types of controlled system components 106 can make use of outputs generated by the EDNN-based algorithms 110, including various types of equipment and other systems associated with one or more of the example use cases described elsewhere herein.

Although the EDNN-based algorithms 110 and the component controller 112 are both shown as being implemented on processing platform 102 in the present embodiment, this is by way of illustrative example only. In other embodiments, the EDNN-based algorithms 110 and the component controller 112 can each be implemented on a separate processing platform. A given such processing platform is assumed to include at least one processing device comprising a processor coupled to a memory.

Examples of such processing devices include computers, servers or other processing devices arranged to communicate over a network. Storage devices such as storage arrays or cloud-based storage systems used for implementation of prediction and remediation database 114 are also considered “processing devices” as that term is broadly used herein.

The network can comprise, for example, a global computer network such as the Internet, a wide area network (WAN), a local area network (LAN), a satellite network, a telephone or cable network, a cellular network such as a 3G, 4G or 5G network, a wireless network implemented using a wireless protocol such as Bluetooth, WiFi or WiMAX, or various portions or combinations of these and other types of communication networks.

It is also possible that at least portions of other system elements such as one or more of the data sources 105 and/or the controlled system components 106 can be implemented as part of the processing platform 102, although shown as being separate from the processing platform 102 in the figure.

For example, in some embodiments, the system 100 can comprise a laptop computer, tablet computer or desktop personal computer, a smartphone, a wearable device, or another type of computer or communication device, as well as combinations of multiple such processing devices, configured to incorporate at least one data source and to execute an EDNN-based algorithm for controlling at least one system component.

Examples of automated remedial actions that may be taken in the processing platform 102 responsive to outputs generated by the EDNN-based algorithms 110 include generating in the component controller 112 at least one control signal for controlling at least one of the controlled system components 106 over a network, generating at least a portion of at least one output display for presentation on at least one user terminal, generating an alert for delivery to at least user terminal over a network, and/or storing the outputs in the prediction and remediation database 114.

A wide variety of additional or alternative automated remedial actions may be taken in other embodiments. The particular automated remedial action or actions will tend to vary depending upon the particular use case in which the system 100 is deployed.

For example, some embodiments implement EDNN-based prediction and remediation algorithms to at least partially automate various aspects of patient care in healthcare applications such as telemedicine. Such applications illustratively involve a wide variety of different types of remote medical monitoring and intervention.

An example of an automated remedial action in this particular context includes generating at least one output signal, such as a prediction visualization signal for presentation on a user terminal, diagnosis information transmitted over a network to a medical professional, and/or prescription information transmitted over a network to a pharmacy or other prescription-filling entity.

Another example of an automated remedial action includes integrating one or more prediction-driven control signals into behavioral and/or wearable technologies for self-intervention. In a more particular example of such an arrangement, control signals generated in system 100 are utilized in a smartphone, wearable or other user device for providing a user with recommendations for behavioral interventions. Another example of such a user device is an interactive smart home assistant, and combinations of these and other user devices may be used in a given embodiment.

Additional examples of such use cases are provided elsewhere herein. It is to be appreciated that the term “automated remedial action” as used herein is intended to be broadly construed, so as to encompass the above-described automated remedial actions, as well as numerous other actions that are automatically driven based at least in part on one or more predictions generated using an EDNN-based prediction algorithm as disclosed herein, with such actions being configured to address or otherwise remediate various conditions indicated by the corresponding predictions.

The processing platform 102 in the present embodiment further comprises a processor 120, a memory 122 and a network interface 124. The processor 120 is assumed to be operatively coupled to the memory 122 and to the network interface 124 as illustrated by the interconnections shown in the figure.

The processor 120 may comprise, for example, a microprocessor, an application-specific integrated circuit (ASIC), a field-programmable gate array (FPGA), a central processing unit (CPU), a tensor processing unit (TPU), a graphics processing unit (GPU), an arithmetic logic unit (ALU), a digital signal processor (DSP), or other similar processing device component, as well as other types and arrangements of processing circuitry, in any combination. At least a portion of the functionality of at least one EDNN or an associated EDNN-based prediction and/or remediation algorithm provided by one or more processing devices as disclosed herein can be implemented using such circuitry.

In some embodiments, the processor 120 comprises one or more graphics processor integrated circuits. Such graphics processor integrated circuits are illustratively implemented in the form of one or more GPUs. Accordingly, in some embodiments, system 100 is configured to include a GPU-based processing platform. Such a GPU-based processing platform can be cloud-based configured to implement one or more EDNNs for processing data associated with a large number of system users. Other embodiments can be implemented using similar arrangements of one or more TPUs.

Numerous other arrangements are possible. For example, in some embodiments, an EDNN and its associated EDNN-based algorithm can be implemented on a single processor-based device, such as a smartphone, client computer or other user device, utilizing one or more processors of that device. Such embodiments are also referred to herein as “on-device” implementations of EDNN-based algorithms.

The memory 122 stores software program code for execution by the processor 120 in implementing portions of the functionality of the processing platform 102. For example, at least portions of the functionality of EDNN-based algorithms 110 and component controller 112 can be implemented using program code stored in memory 122.

A given such memory that stores such program code for execution by a corresponding processor is an example of what is more generally referred to herein as a processor-readable storage medium having program code embodied therein, and may comprise, for example, electronic memory such as SRAM, DRAM or other types of random access memory, flash memory, read-only memory (ROM), magnetic memory, optical memory, or other types of storage devices in any combination.

Articles of manufacture comprising such processor-readable storage media are considered embodiments of the invention. The term “article of manufacture” as used herein should be understood to exclude transitory, propagating signals.

Other types of computer program products comprising processor-readable storage media can be implemented in other embodiments.

In addition, illustrative embodiments may be implemented in the form of integrated circuits comprising processing circuitry configured to implement processing operations associated with one or both of the EDNN-based algorithms 110 and the component controller 112 as well as other related functionality. For example, at least a portion of an EDNN of system 100 is illustratively implemented in at least one neural network integrated circuit of a processing device of the processing platform 102.

The network interface 124 is configured to allow the processing platform 102 to communicate over one or more networks with other system elements, and may comprise one or more conventional transceivers.

It is to be appreciated that the particular arrangement of components and other system elements shown in FIG. 1 is presented by way of illustrative example only, and numerous alternative embodiments are possible. For example, other embodiments of information processing systems can be configured to implement EDNN-based algorithm functionality of the type disclosed herein.

Also, terms such as “data source” and “controlled system component” as used herein are intended to be broadly construed. For example, a given set of data sources in some embodiments can comprise one or more wearable devices of a subject, a smartphone of the subject, and/or one or more sensors associated with the subject. Additionally or alternatively, data sources can comprise video cameras, sensor arrays or other types of imaging or data capture devices. Other examples of data sources include sources of data indicative of online behavior, such as data collected from social media sites and web browsers, or other sources of behavioral data. This includes various types of databases or other storage systems accessible over a network. A wide variety of different types of data sources can be used to provide input data to an EDNN-based algorithm in illustrative embodiments. A given controlled component can illustratively comprise a computer, a smartphone, a wearable device or other type of processing device that receives an output from an EDNN-based algorithm and performs at least one automated remedial action in response thereto.

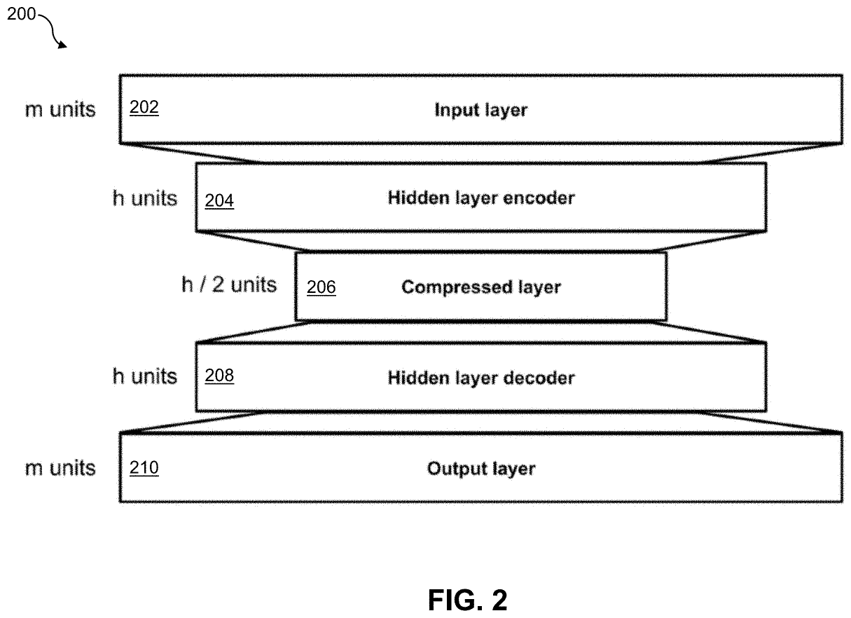

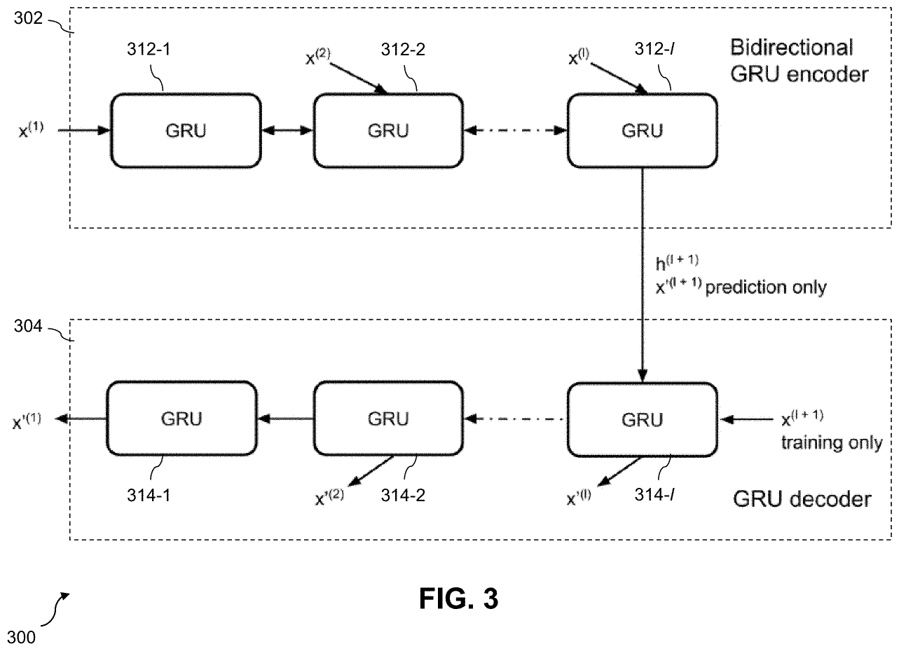

FIGS. 2 and 3 show examples of EDNN architectures that are utilized to implement respective EDNN-based algorithms in illustrative embodiments.

Referring initially to FIG. 2, a portion of an EDNN is shown, implemented utilizing a fully-connected neural network autoencoder architecture 200, also referred to herein as an FNN AD model. The example architecture 200 in this embodiment comprises an input layer 202, a hidden layer encoder 204, a compressed layer 206, a hidden layer decoder 208, and an output layer 210. The hidden layer encoder 204 receives from the input layer 202 an input data subsequence having a relatively high dimension and generates a first intermediate data subsequence having a relatively low dimension for delivery to the compressed layer 206. The hidden layer decoder 208 receives from the compressed layer 206 a second intermediate data subsequence having the relatively low dimension and generates an output data sequence having the relatively high dimension and representing a reconstructed version of the input data subsequence having the relatively high dimension.

In this embodiment, the input layer 202, the hidden layer encoder 204, the compressed layer 206, the hidden layer decoder 208, and the output layer 210 illustratively have respective dimensionalities of m units, h units, h/2 units, h units and m units, respectively. As will be appreciated by those skilled in the art, parameters such as m and h can vary depending on the implementation. In example implementations of such an FNN AD model, m is generally dependent upon the number of features in a specific dataset being used, and in some embodiments was on the order of about 50 to 60, although a wide variety of other values can be used, again depending upon the particular dataset. Values of h such as 10, 20, 30, 40 and 50 were used in some embodiments, and it was found that a value of h=40 provided optimal FNN AD model performance in example schizophrenia-related prediction experiments described herein, although these and other values can be altered in other embodiments. The FNN AD model in some embodiments was trained using an Adam optimizer, with a mean-squared error (MSE) loss function and early stopping, although such training features can also be altered in other embodiments. Those skilled in the art will appreciate that these and other EDNN features can be adjusted as needed to accommodate the particular features and other characteristics of a given implementation.

Turning now to FIG. 3, a portion of an EDNN is shown, implemented utilizing a gated recurrent unit (GRU) sequence-to-sequence architecture 300, also referred to herein as a GRU Seq2Seq model. The example architecture 300 in this embodiment comprises a GRU encoder 302 and a GRU decoder 304. The GRU encoder 302 is illustratively implemented as a bidirectional GRU encoder comprising a plurality of serially-connected GRU cells 312-1, 312-2, . . . 312-l, and having a specified hidden unit size. The GRU decoder 304 is illustratively implemented as a unidirectional GRU decoder comprising a plurality of serially-connected GRU cells 314-l, . . . 314-2, 314-1. The GRU cells 312 are bidirectional GRU cells, and the GRU cells 314 are unidirectional GRU cells, although other arrangements could be used.

The GRU cell 312-1 of the GRU cells 312 in the GRU encoder 302 receives an input data subsequence, which is processed through the GRU cells 312 to generate one or more encoder outputs that are provided as input to the GRU cell 314-l of the GRU cells 314 in the GRU decoder 304. The GRU cells 314 generate at the output of GRU cell 314-1 a reconstructed version of the input data subsequence.

In example implementations of such a GRU Seq2Seq model, values of h such as 10, 20, 30, 40 and 50 were used in some embodiments, and it was found that a value of h=50 provided optimal GRU Seq2Seq model performance in example schizophrenia-related prediction experiments described herein, although these and other values can be altered in other embodiments. The GRU Seq2Seq model in some embodiments had 24 recurrent units (l=24). Other example parameter values used included dropout (rate=0.2) and recurrent dropout (rate=0.2). The GRU Seq2Seq model in some embodiments was trained using an RMSprop optimizer, with an MSE loss function and early stopping, although again such training features can also be altered in other embodiments. As indicated previously, those skilled in the art will appreciate that these and other EDNN features can be adjusted as needed to accommodate the particular features and other characteristics of a given implementation.

Additional details regarding the operation of architectures 200 and 300 in illustrative embodiments are provided elsewhere herein. Their particular configuration as shown in the figures is non-limiting and should be considered illustrative examples only. Numerous other types of EDNNs can be used in other embodiments. Also, other types of machine learning and/or artificial intelligence architectures, illustratively implementing other types of neural networks, can be used in other embodiments. Accordingly, illustrative embodiments herein are not limited to use with EDNNs.

The system 100 can be configured to support a wide variety of distinct applications, in numerous diverse contexts.

For example, illustrative embodiments of the system 100 are configured to predict early warning signs of various behavioral and/or physiological conditions, illustratively using passive sensing data collected with little to no user interaction, from one or more mobile sensors (e.g., a smartphone and/or one or more wearable devices).

In these and other similar use cases, behavioral anomalies are often an early warning sign of mental health deterioration across a variety of conditions, including depression and psychosis. Accordingly, some embodiments disclosed herein predict early warning signs of psychotic relapse from passive sensing data.

Additional details regarding illustrative embodiments will now be described. These illustrative embodiments utilize EDNNs to predict early warning signs of psychotic relapse from passive sensing data, in the context of schizophrenia spectrum disorders or SSDs. SSDs are complex chronic conditions, but the severity of symptomatic experiences and functional impairments vacillate over the course of illness.

Some embodiments disclosed herein provide unobtrusive remote monitoring systems to detect early warning signs of impending symptomatic relapses so as to allow clinicians to intervene before the patient's condition worsens. For example, in a study to be described below, illustrative embodiments predicted a higher rate of anomalies in patients with SSDs within a 30-day near relapse period. Such embodiments can be used to uncover individual-level behaviors that change before relapse, thereby providing technologists and clinicians with unobtrusive digital mental health tools that can predict incipient relapse in SSDs.

SSDs are characterized by a diverse set of symptoms that present themselves heterogeneously throughout the affected population. Symptoms are typically categorized into 2 groups: positive symptoms, which are an exaggeration of normal function (e.g., hallucinations, disorganized speech or thought) and negative symptoms, described as a loss of normal function (e.g., lack of expressiveness, apathy, and asociality). Symptom exacerbation in SSDs leads to a psychotic relapse. Relapse has serious potential consequences, jeopardizing many aspects of patients' lives, including personal relationships and employment, with an increased risk of patients causing harm to themselves or others. Early detection of relapse using the techniques disclosed herein can advantageously inform time-sensitive clinical efforts that may reduce the severity of relapses or prevent their occurrence altogether, thereby significantly reducing direct medical costs of schizophrenia.

The heterogeneity of symptoms and the timing of symptom exacerbation make detecting early warning signs of relapse difficult. Relapse symptoms, unlike common first-episode psychosis symptoms, can appear abruptly. In-depth interviews with patients with SSDs describing their prerelapse symptoms show that symptom manifestation is extremely idiosyncratic but often consistent within individuals. Each individual may have his or her own unique relapse signature, and identifying this signature could be the most effective manner of detecting incipient relapse. Traditional measures of relapse come from clinician-administered rating scales that attempt to quantify a patient's current experience with an SSD. However, it is often unlikely that patients present themselves to a clinician when their symptoms begin to re-emerge or worsen, particularly in an illness characterized by cognitive disorganization, loss of insight, and inconsistent treatment delivery systems where it can be difficult to access care. To prevent symptom exacerbation, illustrative embodiments herein provide tools that are able to detect early warning signs of relapse outside of the clinic.

Over the past decade, improvements in sensing technologies within smartphones, wearables, and other devices have created new opportunities for remote measurement of mental health symptoms. Behavioral data collected with passive sensors from smartphones offer unobtrusive methods to measure trajectories of mental health and mental illness. Smartphones can track a diverse set of behaviors and are owned and utilized by most individuals with SSDs.

For example, some embodiments disclosed herein can utilize one or more aspects of a system referred to as CrossCheck to collect passive sensing data. The CrossCheck system is described in, for example, D. Ben-Zeev et al., “CrossCheck: Integrating self-report, behavioral sensing, and smartphone use to identify digital indicators of psychotic relapse,” Psychiatric Rehabilitation Journal, 40(3), pp. 266-275, September 2017, and R. Wang et al., “CrossCheck: toward passive sensing and detection of mental health changes in people with schizophrenia,” Proceedings of the 2016 ACM International Joint Conference on Pervasive Ubiquitous Computing—UbiComp '16, Heidelberg, Germany, pp. 886-897, September 2016, each incorporated by reference herein in its entirety.

The CrossCheck system combines passive sensing with triweekly self-reported survey measures. Using CrossCheck, researchers were able to predict patient self-reported ecological momentary assessments (EMAs) from passive sensing data and combine both the passive sensing and self-reported data to predict clinician-administered Brief Psychiatric Rating Scale (BPRS) scores. In addition, researchers were able to detect significant changes in patient smartphone social behavior during the 30 days preceding relapse.

Relapse is a rare event, and lack of available data near relapse can make prediction problematic. Anomaly detection is a branch of data mining specifically for the prediction of peculiar, infrequent events. Traditional approaches for anomaly detection within time series involve forecasting and use statistical measures based on cumulative sums, moving averages, and regression models that rely on predicting changes in the underlying distribution of the time series. Forecasting human behavior is an extremely difficult problem, and behavioral data from patients with schizophrenia do not traditionally follow the circadian rhythms seen within a healthy population.

Some embodiments herein utilize EDNN models to identify anomalies in multivariate time series data. Unlike statistical approaches, neural networks do not require assumptions about the underlying distribution of the data and are often ideal compared with classical machine learning techniques because they can provide accurate predictions without the need for complex feature engineering. However, there is a tradeoff. It can be difficult to interpret the reasoning behind why neural networks make specific predictions, leading to the common description that neural networks are black box models. In medicine, specifically, interpretability is important because clinicians need to justify the risk of using new approaches; thus, it is challenging to introduce neural network-based decision making into the clinical workflow.

As will described in more detail below, illustrative embodiments herein successfully implement a neural network based anomaly detection system within the context of behavioral health. One or more such arrangements not only provide accurate detection, but also provide a process for uncovering the underlying behaviors that lead to an anomaly and provide a clinical translation for those behaviors.

Some embodiments herein provide EDNN-based anomaly detection models to predict early warning signs of psychotic relapse using passive sensing data collected from a smartphone. One or more such embodiments are configured to predict early warning signs of relapse using exclusively passive sensing data. The following description of these embodiments includes a post hoc analysis for clinical interpretation of the detected anomalies within the context of SSDs and demonstrates that the disclosed algorithms can detect participant-specific relapse signatures. Additional analysis is provided herein to illustrate how variations in participant data can change model performance.

Example Techniques

CrossCheck System and Study

The CrossCheck system included an Android smartphone application (“app”) combined with a cloud-based data collection and storage platform. The app continuously collected users' passive sensing data and prompted participants every two to three days to self-report EMAs to track both positive and negative symptoms of SSDs. EMAs were not utilized in the example anomaly detection system owing to low completion rates across relapse participants. Table 1 provides an overview of the raw passive sensing data collected using CrossCheck. Sensors also collected environmental data, including ambient sound and light. The ambient sound was utilized by the app to classify when conversations occurred near the participant, but the raw sound and light data were not used. Additional details regarding CrossCheck can be found in the above-cited CrossCheck References.

TABLE 1
Summary of passive sensing behavioral data collected throughout the study.

| Behavior | Description | Derived hourly features |
|---|---|---|
| Acceleration | 3-axis acceleration data were collected | Mean acceleration over the |
| | from a smartphone, sampled from 50-100 | hour |
| | Hz. Raw acceleration features were used | |
| | in order to make the anomaly detection | |
| | system independent of a specific activity | |
| | recognition API platform, such as the | |
| | Android activity recognition API. | |
| App use | Apps running on a user's smartphone were | Number of unique apps opened |
| | recorded every 15 minutes. | within an hour |
| Call | Smartphone calls were tracked, including | Number and duration of |
| | when incoming, outgoing, missed, | incoming, outgoing, missed, |
| | rejected, and blocked calls occurred. | rejected, and blocked calls |
| Conversation | Human voices and conversational episodes | Number and duration of |
| | were detected. | conversations |
| Location | Location information for users was tracked | Time in primary, secondary, |
| | through their smartphones. | and all other locations as well |
| | | as total distance travelled in |
| | | the hour |
| Screen activity | The amount of time users spend on their | Number of times the |
| | smartphones was tracked to learn normal | smartphone was used as well |
| | daily behaviors. This included recording | as the duration of use |
| | the time users' screens were on versus off. | |
| Sleep | On each day, the sleep duration, onset, and | Sleep duration, onset and wake |
| | wake time were detected. These | time. As only the longest |
| | calculations occurred using a combination | sleep episode per day was |
| | of information based upon users' screen | estimated, this is technically a |
| | time, physical activity, ambient sound, and | daily feature. These features |
| | light. | were replicated across all |
| | | hours within a single day. |
| Text | Text messages were tracked, including | Number of received, sent, |
| | when texts were received, sent, drafted, | drafted, outbox, failed to send, |
| | left in a user's outbox, failed to send, and | and queued messages |
| | were queued for sending. | |

The CrossCheck study was a randomized controlled trial (RCT) aimed at testing the efficacy of using passive sensing and self-reported data to identify digital indicators of relapse using the CrossCheck system. The participants enrolled were randomized either into a smartphone group for passive sensing data collection or into a control group to receive treatment as usual. Some embodiments disclosed herein are configured to predict early warning signs of relapse from collected passive sensing data, and so focus exclusively on the smartphone group. Participants enrolled in the study were given an Android smartphone for 12 months and instructed to carry the device with them and complete the EMA. Trained clinical assessors met with participants to conduct a baseline assessment of symptoms and functioning. Clinical assessors also conducted follow-up assessments with participants during months 3, 6, 9, and 12 of the study to administer the 7-item BPRS, which measures psychiatric symptoms associated with SSDs. Participants' electronic medical records (EMRs) were also made available to the clinical assessors. The following events, either reported during assessment or recorded within the EMR, were designated as relapse: psychiatric hospitalization, a significant increase in psychiatric care (including more intensive or frequent services, increased medication dosage, or additional medication prescribed) coupled with an increase of 25% from the baseline total BPRS score, suicidal or homicidal ideation with clinical relevance, self-injury, or violent behavior resulting in harm to another person or property. The date of relapse, any notes surrounding the relapse event, and the reason for designating the event as a relapse were recorded. When corroborating evidence surrounding the relapse was not available within the EMR, clinicians worked with participants during the assessments to gain more information regarding the relapse event.

Relapse is an acute event, but when the early warning signs of relapse begin to surface is an open question. Consistent with previous research on early warning signs of relapse, the 30-day period before relapse was defined as the 30-day near relapse period (NR30), and all data outside of this period were considered days of relative health (DRH).

Study Protocol

The CrossCheck study was approved by the Committee for Protection of Human Subjects at the Dartmouth College and the Institutional Review Board of the Northwell Health System. The study was registered as a clinical trial (NCT01952041).

Participants

Participants were recruited into the RCT from several treatment programs at a psychiatric hospital in New York. Participants were recruited through flyers posted at the study site with the research coordinator's phone number. In addition, researchers reviewed the hospital's EMRs to identify potential participants. A potential participant's clinician was contacted by the investigative team, and after describing the study to the patient, clinicians referred patients interested in the study to the research team.

Eligible participants met the following inclusion criteria: (1) a chart diagnosis of schizophrenia, schizoaffective disorder, or psychosis not otherwise specified, (2) 18 years of age, and (3) an inpatient psychiatric hospitalization, daytime psychiatric hospitalization, outpatient crisis management, or short-term psychiatric hospital emergency room visit within 12 months before beginning the study. Individuals were excluded if they had the following: (1) hearing, vision, or motor impairment that would impede smartphone usage (determined using a smartphone demonstration during screening), (2) a below sixth grade reading level (determined using the Wide Range Achievement Test-4th Edition), and (3) unable to provide informed consent (using a competency screener).

A total of 1367 individuals were initially assessed for eligibility and 149 were enrolled in the study. Eligible individuals who did not enroll were no longer receiving care at the hospital (n=682), failed to meet the diagnostic criteria (n=131), did not want to participate (n=129), or did not meet the severity criteria (n=108). Of the 149 individuals enrolled, 62 were randomized into the smartphone group of the study. Participants included in this example study (n=60) were required to have had at least 10 DRHs collected by the smartphone app.

Feature Extraction and Data Cleaning

An advantage of using neural networks for machine learning is that they have the ability to learn intricate features from raw data. Some embodiments herein exploit this fact by creating features for an EDNN algorithm that were close to the raw data. Hourly features were created from the raw sensor data. A summary of the hourly features used can be found in Table 1. In addition to the passive sensing features, the day of the week and the hour of the day were included as features in the example models. The few features that involved more complex calculations are described below.

Android smartphones track acceleration within a 3D x, y, and z coordinate system. This produces three values for every acceleration reading, namely a=(aₓ, a_{y}, a_{z}). Some embodiments computed the mean hourly acceleration by taking the vector norm of each a within a specific hour and averaging over the values.

Illustrative embodiments also tracked the longitude and latitude locations over time for each participant. The locations for each participant were clustered using the density-based spatial clustering of applications with noise (DBSCAN) algorithm, implemented in the scikit-learn library. DBSCAN clusters samples of high density together; requires a minimum number of samples per cluster, and requires a maximum distance, ε, between points to be specified as hyperparameters. A minimum of 10 samples per cluster were used, and ε was set to 1 km. For each participant, the two majority clusters were tagged as the participant's primary and secondary locations, and all other data points were grouped together into a third cluster. Finally, the distance between each pair of longitude and latitude coordinates was calculated using the Haversine formula. The distances were then summed over each hour.

Two types of missing data were identified. The first type of missing data (type 1) occurred when there was a sensor reading during an hour for one feature but there was no reading within the same hour for another feature. Missing data was imputed for type 1 values with a “0,” indicating a belief that the CrossCheck system was functioning during these hours, but an individual did not partake in specific behaviors that the system records (e.g., no texts were recorded within an hour). A second type of missing data (type 2) was identified during hours where all features were missing. Features were imputed for the second type of missing data utilizing the mean value of a given feature for that hour. Location features (time spent in primary, secondary, and other locations) were filled differently. It was assumed that the participant remained at his or her last recorded location and the features were filled accordingly.

It was also assumed that by using mean filling for type 2 missing values, the anomaly detection models would be directed to focus on finding anomalies within the actual passive sensing data. That being said, missing values, specifically type 2 missing values, could have an implication for function. For example, if a participant stopped using his or her smartphone and the smartphone app, missing values could be an indication of asocial behavior, which may precede relapse. Accordingly, an additional feature was included in the model to indicate the percentage of features filled within a given hour. If this feature was <1, the hour was filled using the type 1 missing data procedure, but if the feature was equal to 1, the hour was filled using the type 2 missing data procedure.

Encoder-Decoder Neural Network Models

Illustrative embodiments herein provide multiple algorithms to detect early warning signs of relapse using passive sensing data. Patients with SSDs are known to not experience normal circadian rhythms that are typically found within a healthy population. Thus, some embodiments apply a neural network approach for multivariate anomaly detection in irregular sensor data. Specifically, illustrative embodiments include a fully-connected neural network autoencoder (FNN AD) model, an example of which is the EDNN architecture 200 shown in FIG. 2, and a gated recurrent unit sequence-to-sequence (GRU Seq2Seq) model, an example of which is the EDNN architecture 300 shown in FIG. 3, both configured to learn to reconstruct an input time series. A GRU network was used in these embodiments instead of a basic recurrent neural network (RNN), or other RNN architectures such as a long short-term memory (LSTM) networks, as the GRU networks counter the vanishing gradient problem that occurs when training basic RNNs, and converge faster during training than LSTM networks. After training the EDNN models, an example EDNN-based algorithm learned participant-specific anomaly thresholds based on the model reconstruction error.

Additional details regarding the example FNN AD and GRU Seq2Seq models of respective FIGS. 2 and 3 will now be described.

Each participant's data was considered to be a time series of varying length L, X={x^{(l)}, . . . , x^{(L)}}, where each x⁽ⁱ⁾ is a multivariate data point, x⁽ⁱ⁾∈R^{m}. By way of example, each x⁽ⁱ⁾ illustratively represented a set of hourly features for a single participant. Subsequences of data of length l were created starting at each i, i={i, . . . , L−l+1}. Note that a given data point, x⁽ⁱ⁾, could be potentially included within each of the 1, . . . , L subsequences. For the FNN AD model, the subsequence length l was set as l=1, and for the GRU Seq2Seq model, the subsequence length was set as l=24. It is to be appreciated that other time series configurations and values can be used in other embodiments.

The models were constructed in the manner illustrated in FIGS. 2 and 3. The FNN AD model was configured in accordance with example EDNN architecture 200 of FIG. 2 and included two fully-connected encoder and decoder layers 204 and 208 that compressed an input subsequence received at input layer 202 to a lower dimension in compressed layer 206 and then recreated the initial subsequence at output layer 210.

For the GRU Seq2Seq model, configured in accordance with the example EDNN architecture of FIG. 3, a subsequence of data is first input into a single encoding layer of a bidirectional GRU encoder 302 with a specified hidden unit size. A bidirectional layer was used for the GRU encoder 302, as bidirectional layers typically improve the results over unidirectional layers. The last GRU cell 312-l in the encoding layer outputs a prediction for the next timestep, x′^{(l+1)} and encodes hidden information from the entire sequence, h^{(l+1)}. This information was then passed as inputs into a unidirectional GRU decoder 304 providing a decoding layer that reconstructed the subsequence in reverse order: {x′^{(l)}, . . . , x′^{(l)}}.

Model Training Procedure

In an example data-splitting and cross-validation procedure, the data for each participant were first split into equal length non-overlapping subsequences, and the subsequences were placed into one of four data sets. Defining NR30 as the 30-day near relapse period and DRH as days of relative health (i.e., all days not in NR30), the data were split into the following:

1. A training data set, comprising only DRH, called H_{R}. These training data are utilized to train each model.

2. A cross-validation data set, comprising only DRH, called H_{CV}. These cross-validation data are utilized to validate the ability of the models to reconstruct sequences of new data.

3. A cross-validation data set, comprising both DRH and NR30, called N_{CV}. These cross-validation data are used to tune the anomaly detection component of the example algorithm as described elsewhere herein.

4. A test data set, comprising both DRH and NR30, called N_{T}. The test data set are used to report the metrics of the anomaly detection algorithm described elsewhere herein.

Experiments were conducted by varying the percentage of relapse participant data to include in each of these four data sets. Specifically, these experiments involved placing different percentages of DRH within H_{R} and H_{CV}, and more particularly placing 0%, 20%, 40%, 60%, and 80% of relapse participants' DRH into H_{R} and H_{CV}. DRH for both relapse and nonrelapse participants were split such that 80% of DRH were placed into H_{R} and 20% into H_{CV}. Nonrelapse participant data were split entirely between H_{R} and H_{CV}.

Monte Carlo cross-validation was used to examine the robustness of the algorithm across different potential N_{CV} and N_{T}. Each Monte Carlo sample was stratified to place equal amounts of NR30 data per participant within N_{CV} and N_{T}. The Monte Carlo procedure was repeated over 100 iterations and the median and IQR of the true-positive rate (TPR or sensitivity), and false-positive rate (FPR) of the current Monte Carlo test set N_{T} were recorded.

Anomaly Detection System. The trained EDNN models were used to reconstruct H_{CV}, N_{CV}, and N_{T}, producing H′_{CV}, N′_{CV}, and APT. For a data point x⁽ⁱ⁾ in each of these data sets and its reconstructed counterpart x′⁽ⁱ⁾, the absolute error of the data points was calculated: e⁽ⁱ⁾=|x⁽ⁱ⁾−x′⁽ⁱ⁾|.

Within the example algorithm, the full-time series was split into subsequences of length l, and any point x⁽ⁱ⁾ could appear in at most 1 different subsequences. Thus, for a point x⁽ⁱ⁾, there can exist l different predictions, {xₗ′⁽ⁱ⁾, . . . , xₗ′⁽ⁱ⁾}. The data set was filtered to include only points that were predicted l times. The error vectors for these data were considered to be normally distributed, e⁽ⁱ⁾˜N(μ, Σ), and the error between H_{CV} and H′_{CV} was used to approximate (μ, Σ) parameterizing the expected error of the example algorithm. An anomaly score s⁽ⁱ⁾∈R was then calculated for error vectors between N_{CV}, N′_{CV}, and N_{T}, N′_{T} using the Mahalanobis distance, which calculates the distance of a point to a distribution as follows: s⁽ⁱ⁾=((e⁽ⁱ⁾−μ)^{T}Σ⁻¹(e⁽ⁱ⁾−μ)^{1/2}.

The average anomaly score for a single day, denoted s^{d}, was calculated from the hourly scores. The Mahalanobis distance from data in N_{CV} was used to optimize an anomaly threshold, τ, for each participant over all s^{d} for that participant. A day was tagged as an anomaly if s^{d}>τ or normal if s^{d}≤τ. The anomaly threshold τ was chosen to maximize the ratio between the TPR divided by the FPR, or TPR/FPR, defining a true positive as an anomaly detected within NR30 and a false positive as an anomaly detected on a DRH. Optimizing this ratio maximized the number of anomalies detected during the NR30 period when minimizing the number of anomalies detected during DRH. This r was applied to the Mahalanobis distances from the held-out test sample, N_{T}, and the final results using the best τ for each participant's N_{T} were recorded.

Evaluation Metrics

The TPR/FPR ratio was used as an evaluation metric to rank model performance. By maximizing this ratio, the sensitivity and specificity of the example models were subsequently maximized. Sensitivity and specificity are metrics commonly used within medicine to assess the strength of a diagnostic test. The sensitivity is equivalent to the TPR and the specificity is equivalent to the true negative rate (or 1−FPR). Thus, by maximizing the TPR/FPR, the example models maximize both sensitivity and specificity.

Anomalies are rare events; thus, it is unlikely that every day within NR30 would contain an anomaly. Clinically, it was assumed that an anomaly detection system for early warning signs of relapse would be relevant as long as the anomalies were rare (low sensitivity and high specificity), but increased (TPR/FPR>1) within NR30. This increased signal could then be used to find passive sensing features that distinguished anomalies within NR30 from anomalies identified within DRH.

Baseline Model and Evaluation

A k-nearest neighbors local outlier factor (LOF) model was used as a baseline comparison against the example neural network models. The LOF model estimated the local density around each data point using a k-nearest neighbor algorithm and then compared the local density of a given data point with the local density of its neighbors. If the point was in a substantially less dense area, it had a higher calculated LOF. An LOF model was initially fit for each relapse participant utilizing H_{R} with the number of neighbors equal to 10, and the number of neighbors was incremented by one until the mean and SD of the LOF scores under H_{CV} converged. The approach described above was then used to calculate anomalies by considering the distribution of LOF scores obtained under H_{CV} and learning an appropriate anomaly threshold for N_{CV}. The LOF model was trained and tested using scikit-learn.

The example neural network models were created using TensorFlow and Keras libraries, and were trained until the validation loss from H_{CV} converged. Cross-validation was used for the example neural network models to determine the optimal hidden layer size (between 10 and 50 units), the percentage of DRH from relapse participant data to include within H_{R} and H_{CV} (between 0% and 80%), and the anomaly threshold τ that maximized the TPR/FPR ratio on N_{CV} (between 0 and 20). For the LOF model, the number of neighbors utilized for the local density within each relapse patient was also optimized.

Two forms of regularization were applied to train the neural networks. For both the GRU Seq2Seq and the FNN AD models, early stopping was used to terminate model training when the reconstruction error from H_{CV} increased. In addition, dropout (rate=0.2) and recurrent dropout (rate=0.2) were applied to the GRU Seq2Seq model. Dropout masks, or drops, inputs randomly within the network, whereas recurrent dropout adds this mask between the recurrent layers at each timestep. This exposed the trained network to different permutations of the training data to prevent overfitting. Batch normalization was briefly used during model creation, but it was found that batch normalization did not improve anomaly detection performance and therefore was not used to train the final iteration of the models.

Results

Data Overview

A total of 20,137 days of mobile sensing data were collected from 60 patients with SSDs. Relapse events were recorded for 18 of 60 participants (30%) during the 1-year study, totaling 726 days of data collected within any NR30 data (0.037% of the total days of data collected). Table 2 provides a summary of the data collected from the relapse and nonrelapse groups.

TABLE 2
Summarized data characteristics for relapse and nonrelapse participants
(continuous characteristics listed by median (IQR)).

| Characteristics | Relapse | Nonrelapse |
|---|---|---|
| Patients, n | 18 | 42 |
| Age at beginning of study (years), | 33 (23-47) | 40 (26-50) |
| median (IQR) | | |
| Female, n (%) | 8 (44) | 17 (40) |
| Number of days of data collected | 335 (285-346) | 295 (176-361) |
| per participant, median (IQR) | | |

Missing hours of data (type 2), median (IQR)

| | | |
|---|---|---|
| Number of hours | 2309 (1333-2551) | 1785 (660-2871) |
| Percentage of total hours | 25.73 (14.77- | 27.17 (7.72-52.50) |
| | 28.73) | |

Diagnosis, n (%)

| | | |
|---|---|---|
| Schizophrenia | 9 (50) | 17 (40) |
| Schizoaffective disorder | 7 (39) | 18 (43) |
| Psychosis not otherwise | 2 (11) | 7 (17) |
| specified (NOS) | | |

Assessment at baseline, median (IQR)

| | | |
|---|---|---|
| BPRS (7-item) total | 29 (23-33) | 24 (21-29) |

Lifetime hospitalizations, n (%)

| | | |
|---|---|---|
| 1-5 | 13 (72) | 30 (71) |
| 6-10 | 1 (6) | 8 (19) |
| 11-15 | 1 (6) | 3 (7) |
| 16-20 | 1 (6) | 0 (0) |
| 20+ | 1 (6) | 1 (2) |
| Missing or declined | 1 (6) | 0 (0) |

Distribution of relapse events, n (%)

| | | |
|---|---|---|
| 1 relapse event | 14 (78) | N/A |
| 2 relapse events | 1 (5) | N/A |
| 3 relapse events | 3 (17) | N/A |

Anomalies Increased Near Relapse

The highest performing cross-validation results for each model, with hyperparameters, are shown in Table 3. All results are listed using median (IQR) sensitivity and specificity. Across all model architectures, the FNN AD model using 80% of the data from DRH with 40 hidden units had the highest rank across participants (9.28), achieving a median sensitivity of 0.25 (IQR 0.15-1.00) and specificity of 0.88 (IQR 0.14-0.96). LOF models did not show predictive power (sensitivity 1.0 and specificity 0.0) and were not included in the results.

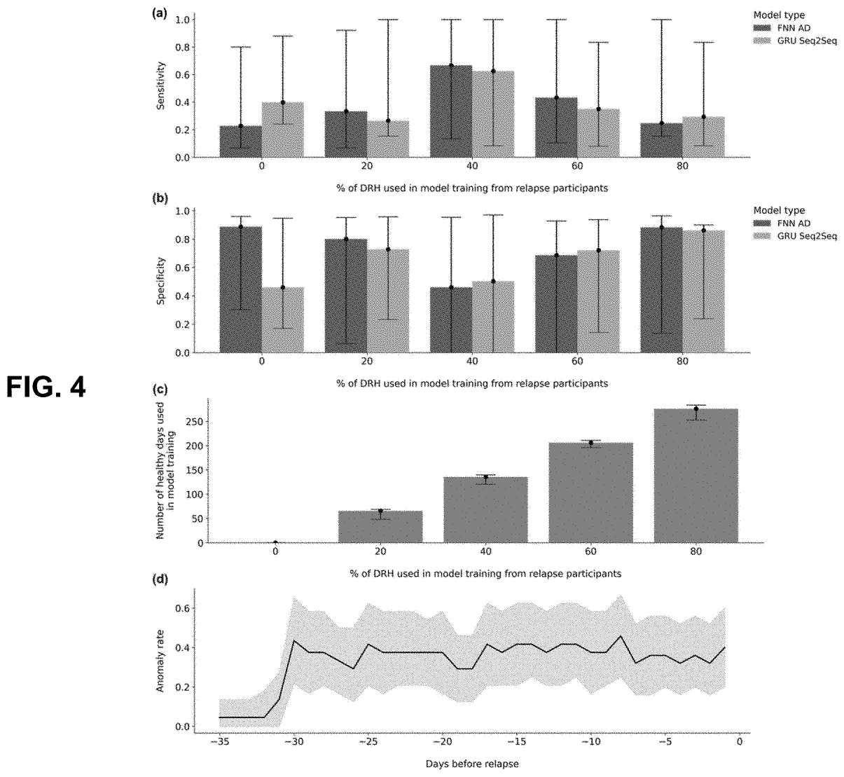

FIG. 4 shows the resulting sensitivity and specificity achieved from models trained on different percentages of DRH.

More particularly, this figure shows the overall model results, including the anomaly rate of the best performing model across the NR30 and in parts (a) to (c) split by the DRH used in model training. In parts (a) to (c), the bar heights describe the median value of the metric listed on the y-axis across study participants and the error bars show lower and upper quartile values (25% and 75% percentiles of the data). In parts (a) and (b), LOF models are not shown as they did not hold predictive power. Part (a) shows sensitivity, or true positive rate, of the models, part (b) shows specificity, or true negative rate, part (c) shows median number of DRH used to train each model from each study participant, and part (d) shows average (solid line) and 95% CI (gray shading) anomaly rate across relapse participants beginning 35 days before relapse using the best performing model (fully-connected neural network autoencoder, 80% of DRH in train, 40 hidden units).

The data presented in FIG. 4 shows that adding a larger percentage of DRH to model training initially increased the sensitivity and decreased the model specificity, but then decreased the sensitivity and increased the specificity as more data were added. The data also shows that the anomaly rate increased before the NR30 period but then remained fairly constant among participants.

TABLE 3
Cross-validation results per model type within relapse
participants listed by median (IQR).

| | | Days of | | Sensitivity, | Specificity, |
|---|---|---|---|---|---|
| | | relative health | Hidden | median | median |
| Model | Rank | in train, % | units | (IQR) | (IQR) |
| FNN AD | 9.28 | 80 | 40 | 0.25 | 0.88 |
| | | | | (0.15-1.00) | (0.14-0.96) |
| GRU | 12.72 | 80 | 50 | 0.29 | 0.86 |
| Seq2Seq | | | | (0.08-0.83) | (0.24-0.90) |

Anomaly Detection System Identified Specific Near Relapse Behaviors Individuals often report symptom exacerbation, which could be used to predict the onset of relapse. Identifying participant-specific behaviors that are consistent during relapse would give clinicians a potential signature to identify when a patient needs clinical support. A total of four participants within the study relapsed multiple times. A post hoc analysis was performed using the best-performing example algorithm across participants (FNN AD, 80% of DRH in train, hidden unit size=40) to compare features on NR30 anomalous days with DRH within multirelapse participants. Cohen's d was used to calculate the effect of continuous features on discriminating an NR30 anomaly to any DRH and the OR for calculating whether type 2 missing data appeared more frequently in NR30 anomalies.

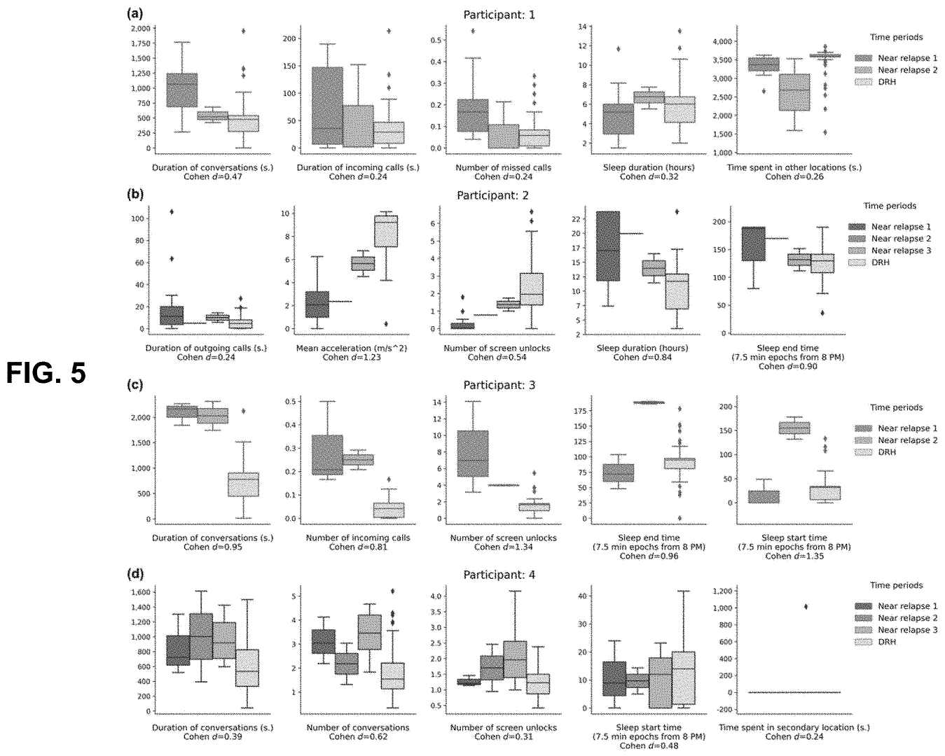

FIG. 5 shows the distribution of the five features with the largest effect per participant on differentiating detected anomalies within NR30 from DRH. More particularly, this figure shows the hourly features that had the greatest effect on differentiating identified anomalous days near relapse (NR30) from all DRH within the four multirelapse participants. The Cohen's d was used to identify the five features that were the most differentiated. Each subfigure, (a) to (d), displays boxplots comparing the distribution of these features on anomalous days within each NR30 period compared with all DRH. The center line in the boxplot is the median value, the box limits are the IQR, and the whiskers are 1.5× the IQR. Points outside of the whiskers are greater than or less than 1.5× the IQR. A lower IQR signifies that the median result is more generalizable. For example, in part (a), anomalies were identified within two NR30 periods, described in the figure as Near relapse 1 and Near relapse 2. The two left boxes on each plot show the distribution of the feature for anomalies detected within each of these two NR30 periods and the right box shows the distribution of this feature on all DRH outside of the two NR30 periods.

Notes surrounding each relapse, extracted from the participant's EMR or obtained during clinical visits, were compiled by a team of trained clinical assessors. These notes were used as a qualitative validation to understand whether the identified features from the analysis were presented to clinicians. The results of each comparison are now described for features that were identified to have a large effect (Cohen's d>0.8), medium effect (0.5≤Cohen's d<0.8), or the feature with the largest effect if no features with a large or medium effect were identified. The ORs indicated that type 2 missing data did not discriminate anomalies within NR30 from DRH for multirelapse participants (OR<1 for all multirelapse participants). The particular features are described in detail elsewhere herein.

Multirelapse Participant 1

No feature with a large or medium effect was identified for this participant. The conversation duration had the largest effect (Cohen's d=0.47), which increased before relapse, as shown in FIG. 5. Clinical notes from the first relapse event indicate that the participant was hospitalized because she was tired of hearing voices, which suggested that her neighbors were constantly talking about her. Notes from the second relapse did not describe any participant behavior.

Multirelapse Participant 2

The participant's mean acceleration (Cohen's d=1.23), sleep end time (Cohen's d=0.90), and sleep duration (Cohen's d=0.84) had a large effect. FIG. 5 shows that the mean acceleration decreased in all three NR30 periods for this participant, whereas the sleep duration and end time increased. Clinical notes from the first relapse identified that the participant had been feeling ill, specifically that his “brain was shaking.” On the second relapse, the participant stated that he felt like he “was going to die,” and was feeling depressed. The clinician wrote that the patient “has had difficulty sleeping.” Notes regarding the third relapse indicate that the participant had been disorganized, physically aggressive toward his mother, and was barely sleeping.

Multirelapse Participant 3

The participant's sleep start time (Cohen's d=1.35), number of smartphone screen unlocks (Cohen's d=1.34), sleep end time (Cohen's d=0.96), duration of conversations (Cohen's d=0.95), and number of incoming calls (Cohen's d=0.81) had a large effect. FIG. 5 shows abnormal behavior in the sleep start and end times for all relapse periods, but is inconsistent in the direction of how the behavior differs from the median value across each relapse. The number of screen unlocks, incoming calls, and duration of conversations increased in both relapse periods. Notes regarding the first relapse did not identify any specific behavioral changes. Clinical notes from the second relapse identified that the participant had been spending his days “making music and beats” and was sleeping less at night, but had increased sleep during the day. The notes also identified the participant as having auditory hallucinations.

Multirelapse Participant 4

One feature, the number of conversations, had a medium effect (Cohen's d=0.62) for this participant. FIG. 5 shows that the duration of conversations increased during all three relapse periods. Notes from the first relapse did not describe any specific behavioral differences in the participant. Clinical notes from the second relapse indicated that the participant presented herself to outpatient psychiatry with “signs of catatonia” and that the participant had mostly stopped speaking, although she had occasional spontaneous speech. Notes were not available regarding the third relapse event.

Anomalies Contained Fewer Hours of Type 2 Missing Data

It was found that type 2 missing data did not have an effect on distinguishing anomalies within NR30 for the four multirelapse participants. This question was examined more broadly to determine how missing data influenced all detected anomalies. A one-sided Mann-Whitney U test was conducted to test the following hypothesis: predicted anomalies contain a smaller number of type 2 hours filled compared with all other days. Individual participant factors were controlled for using participant-specific Mahalanobis distance thresholds for anomaly designation. Anomalies had a median of 0 (IQR 0-6) hours of data filled using the type 2 missing data procedure, and all other days had a median of 2 (IQR 0-16) type 2 hours of data filled. The one-sided test was significant (U=514,546; P<0.001), indicating that anomalous days were significantly less likely to contain type 2 missing data.

Variations in Relapse Participant Data Affected the Quality of Anomaly Detection

The participant-level anomaly detection results were analyzed to determine how variations in data quality affect the generalizability of the example models. Table 4 summarizes the results of using linear regression to assess the significance between the sensitivity and the specificity of the highest performing model (FNN AD, 80% of DRH in train, hidden unit size=40) and the data quality parameters. All data quality parameters were significant (P<0.001). Increasing the number of days of raw data and the percentage of days within NR30 increased the sensitivity of the model (β=0.60, 95% CI 0.48 to 0.72; β=0.73, 95% CI 0.49 to 0.97) but decreased the specificity of the model (β=0.69, 95% CI −0.81 to −0.57; β=−0.71, 95% CI −0.95 to −0.47). Increasing the number of days per NR30 period and the number of relapse events decreased the sensitivity of the model (β−0.43, 95% CI −0.52 to −0.34; β=−0.82, 95% CI −1.02 to −0.62) but increased the specificity of the model (β=0.33, 95% CI 0.23 to 0.43; β=0.87, 95% CI 0.67 to 1.07).

TABLE 4
Linear regression results between sensitivity and
specificity and different data parameters.

| | Sensitivity | | Specificity | |
|---|---|---|---|---|
| | | P | | P |
| Parameters | Coefficient β | value | Coefficient β | value |
| Days of raw data | .60 (95% CI 0.48 to | <.001 | −.69 (95% CI −0.81 to | <.001 |
| | 0.72) | | −0.57) | |
| Days per near relapse | −.43 (95% CI −0.52 to | <.001 | .33 (95% CI 0.23 to | <.001 |
| period | −0.34) | | 0.43) | |
| Percentage of days near | .73 (95% CI 0.49 to | <.001 | −.71 (95% CI −0.95 to | <.001 |
| relapse | 0.97) | | −0.47) | |
| Relapse events | −.82 (95% CI −1.02 to | <.001 | .87 (95% CI 0.67 to | <.001 |
| | −0.62) | | 1.07) | |
| Intercept | .00 (95% CI −0.04 to | >.99 | .00 (95% CI −0.04 to | >.99 |
| | 0.04) | | 0.04) | |

Principal Findings

In the example study described above, a model was created, exclusively using passive sensing data from a smartphone, to predict behavioral anomalies that could indicate early warning signs of psychotic relapse. Developing an anomaly detection system from exclusively passive sensing data requires minimal effort for data collection from the participant and could lead to more objective and unobtrusive ways of monitoring symptoms of SSDs. The example anomaly detection system achieved a median sensitivity of 0.25 (IQR 0.15-1.00) and specificity of 0.88 (IQR 0.14-0.96; a 108% increase in anomalies near relapse), indicating that anomalies increased before relapse but were restricted to specific days within the defined NR30 period. Once anomalous days were identified within NR30, it was demonstrated that the disclosed methodology can be used to identify participant-specific behavioral signatures that occur across multiple NR30 periods. In other embodiments, anomaly detection models can be used to identify days that contain these signatures and supervised learning approaches could then be deployed to detect these signals as early warning signs of relapse. Identifying patient-specific behaviors that change exclusively before relapse could provide clinicians an indicator to measure when patients are declining in health and create time for early intervention.

Anomalies increased within NR30, but with low sensitivity. This low sensitivity could be owing to the choice of a NR30 period or the use of the TPR/FPR ratio as a validation metric for the example study. A NR30 period was chosen because early warning signs of relapse might begin to develop up to one month before the actual relapse event. The low sensitivity indicated that only specific days within this 30-day period were considered anomalies, and training algorithms to target these specific days could increase sensitivity. Another approach to increase sensitivity would be to shorten the number of days included in the near relapse period. For example, a 14-day near relapse period could be used. In illustrative embodiments, an increased anomaly rate 30 days before relapse was observed, as shown in FIG. 4, which remained fairly constant; therefore, shortening the near relapse period was not further investigated. The example algorithms in these embodiments may result in a constant anomaly rate during a near relapse period of any prespecified length as these algorithms are trained to look specifically for behavioral differences within these periods.

In addition, the TPR/FPR ratio was used for model selection rather than directly optimizing for sensitivity or specificity. Most machine learning algorithms use the area under the receiver operating curve to assess the predictive power of a model, although this approach was not used in the example anomaly detection algorithm. Anomaly detection, by definition, searches for extremely rare events. To introduce this process into a clinical workflow, a balance can be struck between highlighting potential early warning signs of relapse without overburdening the clinician and health care system with a high anomaly rate. This can be achieved using the disclosed example modeling approach as an increase in anomalies before relapse was shown without sacrificing the specificity of the results.

To increase model sensitivity in this context, additional or alternative behaviors can be used to identify early warning signs of psychotic relapse. A process can then be created where anomaly detection is first used to identify candidate relapse signatures and then supervised learning algorithms are trained to identify these signatures. This would, in turn, limit the feature space to the behaviors per individual that were differentiated before relapse. In addition, identifying these specific signatures as a starting point for a positive signal would allow clarification as to whether false positives were merely noise or hold clinical significance. In this example study, a relapse event was indicated for most of the participants by either a psychiatric hospitalization or a significant increase in symptoms as reported by clinician-administered BPRS. It is possible that symptoms were exacerbated on days outside of NR30, and the example system detected these days as anomalies. This symptom exacerbation was not given clinical oversight; thus, there was no available mechanism to validate whether these anomalies should be considered true positives.

Days that contained more hours of type 2 missing data, in which no passive sensing data for the entire hour existed, were significantly less likely to be tagged as anomalies. The example approach to using mean filling for type 2 data was based on two assumptions: (1) that behavioral features collected from passive sensors should be prioritized for anomaly detection and (2) that it is possible that a large quantity of missing data might be a sign of asocial behavior and this should be accounted for with an additional feature that tracks the amount of data missing over an hour. It may be assumed that imputed data points should not be detected as anomalies, particularly if the imputed values are located in high-density regions of the feature distribution. In some embodiments, anomalies were significantly less likely to contain missing values (P<0.001), indicating that the Mahalanobis distance per individual was less for imputed hours and anomalies were more likely to include non-type 2 data points. It is possible that the potential effect of missing data on relapse was ignored. For example, the missing data feature did not distinguish anomalies within NR30 in the four multirelapse participants.

It was observed that increasing the amount of relapse participant data used for model training did not always increase the resulting sensitivity and specificity. FIG. 4 shows that the performance of the example models increased in sensitivity and decreased in specificity when the percentage of DRH from relapse patients used in model training was increased from 0% to 40%. A reverse trend (decreased sensitivity, increased specificity) was observed when the amount of DRH was increased from 40% to 80%. This demonstrates that as models learned participant-level behaviors, there was a threshold for the amount of data required for model training (approximately 135 days from data from FIG. 4) before a model could begin to distinguish anomalous behaviors within NR30. It was also found that the example anomaly detection system is sensitive to the quality of the relapse participant data. Table 4 demonstrates that increasing the total percentage of NR30 days increased the sensitivity (β=0.73), but not if this increased the average number of days within NR30 (β=−0.43), and increased the total number of relapse events (β=−0.82). Subsequently, having a higher number of relapse events increased (β=0.87) the specificity of the model, but not if this increased the percentage of days within NR30 (β=−0.71), and increased the number of days of raw data (β=−0.69). Taken together, the results depended on identifying homogeneous behavioral signals that occurred exclusively in NR30.

Given the importance of finding homogeneity in the signal, an examination was made as to whether consistent signals could be uncovered in participants that might indicate SSD symptom exacerbation. It can be difficult to introduce neural network models within clinical practice owing to their black box nature, even though they can achieve higher performance than classical machine learning models. In some embodiments disclosed herein, interpretability is critical because clinicians need to develop an understanding and trust of an algorithm's decision-making process. A post hoc notion of interpretability was used to identify participant-specific features that differed during NR30 anomalies. The effect size, a metric traditionally used to measure the strength of a treatment in an RCT, was chosen to identify the most differentiated features within NR30. Features were identified with a medium to large effect (Cohen's d≥0.5) in three of four multirelapse patients. The identified features encompass different aspects of social behavior, sleep, and physical activity.

With regard to interpreting the behavior changes identified within NR30, smartphone social behavior tends to decrease across participants before relapse. Accordingly, smartphone social behavior was examined at the individual level. Multirelapse participant 3 exhibited increased smartphone social behavior before relapse, but, from contextual notes, it was discovered that this participant experienced auditory hallucinations, potentially explaining the increased conversation duration detected by the smartphone as well as other increased smartphone social behaviors found. Multirelapse participant 4's conversation duration increased with a medium effect (Cohen's d=0.62), contrasting the physician's notes, which stated that the participant was barely speaking and potentially catatonic. FIG. 5 shows that the elevated conversation signal, whether from the participant or the environment, was unique to anomalies within each of the three NR30 periods.

In addition, it was detected that changes in sleeping behavior had a large effect on two participants and decreased acceleration had a large effect on one participant. Previous research has shown that patients with SSDs are at a significantly higher risk of developing a sleep disorder or worsened sleep near relapse. Multirelapse participant 2's detected sleep duration increased before relapse. In addition, the participant's acceleration decreased. Social withdrawal and physical inactivity are common symptoms of SSDs. These symptoms interfere with functioning, potentially leading to relapse, and relapse can produce aggression. The symptoms identified were consistent with the clinician's explanation of the second relapse event for this participant, which described changes in sleep and aggressive behavior. Thus, the features that were most differentiated are consistent with past research identifying early warning signs of relapse.

Although differentiated features for each participant were found that were consistent with the notes surrounding relapse, the changes detected by the passive sensors were not always consistent with the changes described in the clinical notes. For example, it was found that participant 2's sleep increased before relapse, with a large effect, whereas the clinician's notes stated that sleep decreased. Similarly, for participant 4, the number of conversations increased before relapse with a medium effect, whereas the clinician's notes stated that the participant exhibited signs of catatonia. The smartphone algorithms used to detect conversation relied on ambient sound to detect human voice and conversational exchanges, but do not necessarily detect the voice of the participant. In addition, the sleep algorithm used detected sleep based on a combination of smartphone usage, ambient light, stationary behavior, and environmental silence, all features that might occur when someone is still but not necessarily sleeping. When interpreting the result of a black box algorithm clinically, the algorithm's results should be interpreted in the context of the technical capabilities of the passive sensing system used before judging the outputs of the system literally. Thus, although smartphones can find meaningful relapse signatures, the interpretations of these signatures should be corroborated with the patient and other qualitative information to better understand the underlying behaviors that preceded relapse.

As indicated previously, the example study had a limited sample size. The study consisted of 60 participants with SSDs, including 18 participants who relapsed. Most participants did not relapse at multiple points throughout the study and it was not possible to assess whether the features underlying anomalies were consistent with relapse for those participants. However, the example study demonstrated the viability of utilizing anomaly detection to predict early warning signs of relapse exclusively from smartphone behavior.

Example Relapse Prediction System Illustrative embodiments herein provide a clinical intervention system to predict early warning signs of relapse that can be deployed within the clinical workflow. Although the possibility of clinicians having patient data at their fingertips seems appealing, it is also a liability for clinicians if they have 24/7 monitoring capabilities and choose not to act when a patient is potentially in danger. One possible solution to this issue is to introduce a clinical technology specialist into a patient's care team whose responsibility is to successfully introduce and maintain technology-based services within the clinic. It is evident that there is a gap between technology intervention creation and implementation. Overall, acceptability will continue to play a large role in implementing such interventions.

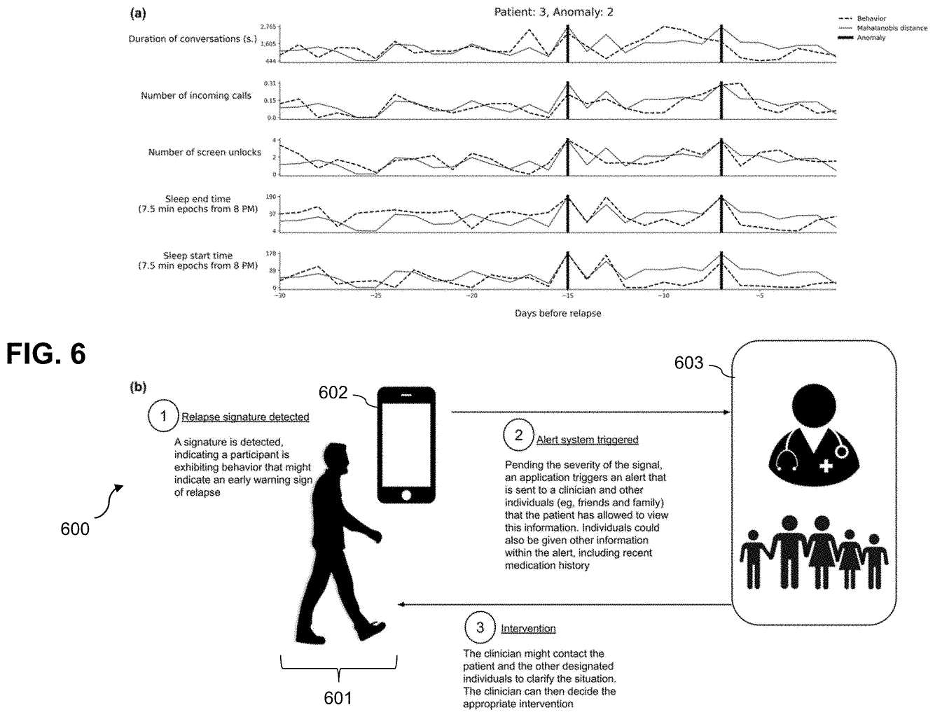

FIG. 6 shows an example of an anomaly detection system 600 with clinical intervention functionality in an illustrative embodiment. The anomaly detection system 600 includes a smartphone 602 of a user 601. The smartphone 602 communicates over one or more networks, not shown, with one or more other processing devices 603 associated with a clinician and other individuals. The smartphone 602 in this embodiment may be viewed as an example implementation of processing platform 102, one or more data sources 105 and one or more controlled system components 106.

The figure includes parts (a) and (b). Part (a) of the figure shows the variations in the behavioral features detected by the smartphone 602 over time. The features in this example include duration of conversations, number of incoming calls, number of screen unlocks, sleep start time and sleep end time, the latter two measured in 7.5 minute epochs from 8 PM. The dashed black lines in part (a) of the figure each represent an hourly feature trajectory from the anomaly detection system 600, as identified on the y-axis, during NR30. The solid gray line on each plot is the Mahalanobis distance, which can be interpreted as an anomaly score that increases as the likelihood of detecting an anomaly increases. The two vertical thick black lines on each plot are detected anomalies.

Part (b) of the figure shows an example of how anomaly detection information generated by the smartphone 602 can be utilized by a clinician and/or other individuals designated by the patient to intervene during symptom exacerbation. Responsive to one or more detected anomalies, the smartphone 602 sends one or more alerts to the one or more processing devices 603. The anomaly detection system 600 is illustratively configured to send such alerts from the smartphone 602 to the one or more processing devices 603 only when a patient is in crisis so as not to overburden the clinician and the healthcare system.

An example process illustrated in part (b) of the figure more particularly comprises steps 1 through 3, which are as follows, although it is to be appreciated that additional or alternative steps can be used in other embodiments. 1. Relapse signature detected. A relapse signature is detected utilizing an app on smartphone 602 that implements EDNN protection and remediation algorithms of the type described elsewhere herein. The detected relapse signature in some embodiments can comprise a single detected anomaly of a particular type, or a particular sequence or other arrangement of multiple detected anomalies. The detected relapse signature indicates that the user 601 is exhibiting behavior that might indicate an early warning sign of relapse.

2. Alert system triggered. The detection of the relapse signature in the app on the smartphone 602 causes the app on the smartphone 602 to trigger the execution of at least one automated remedial action relating to the user 601. In this example, the automated remedial action includes the generation of an alert that is transmitted by the smartphone 602 over one or more networks to the one or more processing devices 603. The generation of the alert is illustratively controlled based at least in part on the severity of a signal characterizing the detected relapse signature. The alert is illustratively sent to a clinician and/or other individuals (e.g., friends and family) that the patient has previously designated as permitted to view this information. Such individuals can also be given other information within or otherwise associated with the alert, such as recent medication history.

3. Intervention. Responsive to receipt of the alert, the clinician and/or other individuals associated with respective ones of the one or more processing devices 603 illustratively contact the patient to clarify the situation. For example, in some embodiments, the clinician can contact the patient and contact one or more other individuals, although numerous alternative arrangements are possible. The clinician and/or the other individuals can then decide the appropriate intervention.

It is to be appreciated that these example process steps and other process steps disclosed herein can be varied in other embodiments. For example, as indicated previously, additional or alternative steps may be used, and one or more steps may at least partially overlap with one or more other steps.

The FIG. 6 embodiment provides an example framework for an SSD behavioral monitoring and intervention system utilizing anomaly detection of the type disclosed herein. In other embodiments, variations in this example framework can be made in terms of, for example, the level of patient interaction with the system, how and when detected anomalies are presented to the clinician or other relevant parties, and a defined procedure that an individual should take to intervene in care when anomalies are detected. These and other embodiments disclosed herein provide systems for detecting early warning signs of psychotic relapse, suitable for deployment within a clinical workflow.

Illustrative embodiments disclosed herein can predict behavioral anomalies preceding relapse events recorded with and without BPRS. Additionally or alternatively, some embodiments can be used to predict an increase in anomalies before relapse, utilizing features derived from passive sensing data exclusively, thereby providing an anomaly detection system that does not rely on patient self-reporting.

Other embodiments can be configured to identify early warning signs of relapse across larger and more diverse patient populations with SSDs. These approaches could be tested across different smartphone passive sensing apps such that they become platform independent. In addition, models can be trained to detect patient-specific relapse signatures, which could increase model sensitivity. Finally, tools can be codesigned with clinicians and patients for remote monitoring of SSD symptoms.

In some embodiments, example anomaly detection models are provided using EDNNs to predict early warning signs of psychotic relapse. The example models predicted an increase in anomalies within the 30-day period preceding relapse. These embodiments also provide a methodology to uncover behaviors that change before relapse, which could be used to identify patient-specific relapse signatures. Some implementations are in the form of a remote monitoring system for SSDs.

These and other embodiments disclosed herein advance the field of digital mental health to create effective remote monitoring systems for serious mental illness and other behavioral and/or physiological issues.

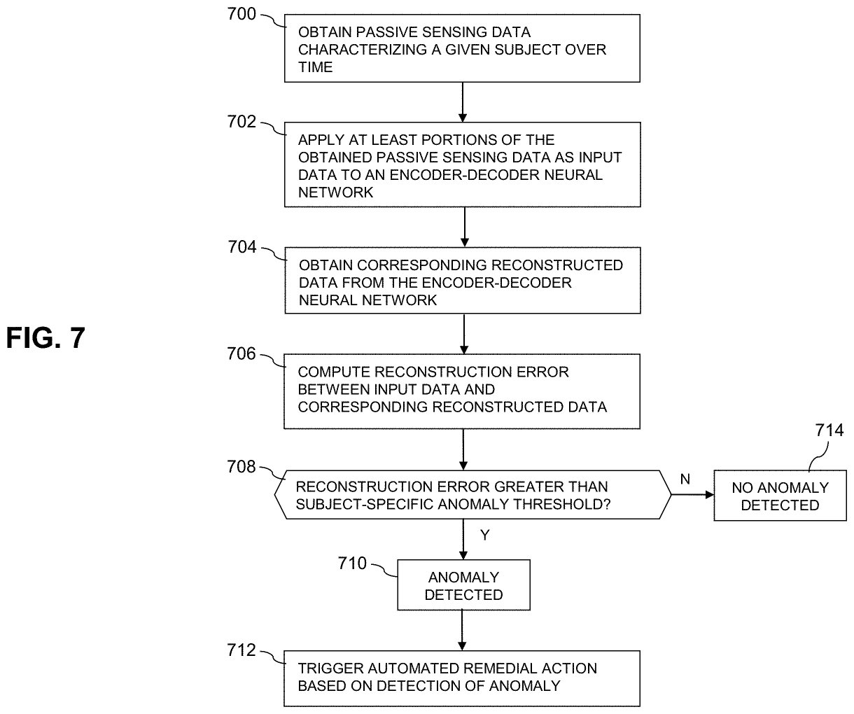

FIG. 7 shows an example process utilizing an EDNN adapted to prediction in contexts such as behavior and/or physiology in an illustrative embodiment. The process as shown includes steps 700 through 714, and is suitable for use in the system 100 but is more generally applicable to other types of systems comprising at least one processing device configured to perform EDNN-based prediction and remediation.

The steps of the FIG. 7 process are illustratively performed at least in part by or under the control of a processing platform such as the processing platform 102 in the system 100. Other arrangements of additional or alternative system components can be configured to perform at least portions of one or more of the steps of the FIG. 7 process in other embodiments. The FIG. 7 process generally provides an example algorithm implemented by at least one processing device for EDNN-based prediction and remediation. Accordingly, the FIG. 7 process can be viewed as one possible implementation of the one or more EDNN-based algorithms 110 of processing platform 102 in the FIG. 1 embodiment.

In step 700, passive sensing data is obtained, characterizing a given subject over time. For example, the passive sensing data may be obtained from a smartphone, a wearable device and/or one or more other devices associated with the subject.

In step 702, at least portions of the obtained passive sending data are applied as input data to an EDNN, illustratively configured in accordance with an EDNN model such as the fully-connected autoencoder model of FIG. 2 or the GRU sequence-to-sequence model of FIG. 3.

In step 704, corresponding reconstructed data is obtained from the EDNN.

In step 706, reconstruction error is computed between the input data and the corresponding reconstructed data.

In step 708, a determination is made as to whether or not the reconstruction error is greater than a subject-specific anomaly threshold. Responsive to an affirmative determination, the process moves to step 710, and otherwise moves to step 714.

In step 710, an anomaly is detected.

In step 712, at least one automated remedial action is triggered responsive to the detected anomaly. The current iteration of the process then ends, at which the point the process illustratively returns to step 700.

In step 714, no anomaly is detected. The current iteration of the process ends, at which the point the process illustratively returns to step 700.

The steps of the FIG. 7 process are illustratively repeated for one or more iterations, for example, at predefined intervals, substantially continuously throughout a given period of time, and/or under other conditions.

The particular processing operations and other system functionality described in conjunction with the flow diagram of FIG. 7 are presented by way of illustrative example only, and should not be construed as limiting the scope of the disclosure in any way. Alternative embodiments can use other types of processing operations involving EDNN-based prediction and remediation algorithms in behavioral and/or physiological contexts. For example, the ordering of the process steps may be varied in other embodiments, or certain steps may be performed at least in part concurrently with one another rather than serially. Also, one or more of the process steps may be repeated periodically, or multiple instances of the process can be performed in parallel with one another in order to implement a plurality of different EDNN-based prediction and remediation arrangements for multiple distinct subjects having respective distinct subject-specific anomaly thresholds.

Functionality such as that described in conjunction with the flow diagram of FIG. 7 can be implemented at least in part in the form of one or more software programs stored in memory and executed by a processor of a processing device such as a computer or server. As indicated elsewhere herein, a memory or other storage device having executable program code of one or more software programs embodied therein is an example of what is more generally referred to herein as a “processor-readable storage medium.”

It is to be appreciated that the particular systems, use cases, features and functionality described above are examples only, intended to demonstrate utility of illustrative embodiments, and should not be viewed as limiting in any way.

As is apparent from the foregoing description, illustrative embodiments disclosed herein have specific application towards mental and behavioral health monitoring in humans. For example, some embodiments are configured to input behavioral features from humans (e.g., sleep, activity, etc.), collected from personal devices (e.g., smartphones, wearables, etc.). The behavioral features are reconstructed by an EDNN model. The error between the reconstructed features and actual features are calculated, and the error is thresholded to designate an anomaly. Such detected anomalies illustratively have implications relating to health changes. This is an example of an embodiment that implements anomaly detection, with the reconstruction outputs of the decoder being compared against the original input, and the resulting reconstruction error being thresholded to identify anomalies.

Accordingly, illustrative embodiments disclosed herein can be advantageously configured to compare a reconstructed input generated by an EDNN with the actual input, and threshold on the error to identify anomalies.

Such embodiments are illustratively implemented in the form of anomaly detection systems, where a given such system predicts deviations from a “normal state,” with potential implications towards change in disease state. Numerous other prediction arrangements can be used in other embodiments.

Different embodiments can utilize different types of EDNNs, such as the fully-connected autoencoder and the GRU sequence-to-sequence models described previously. It should be noted, however, that a wide variety of other types of machine learning or artificial intelligence arrangements can be used in other embodiments.

Automated remedial actions taken based on outputs generated by an EDNN-based algorithm of the type disclosed herein can include particular actions involving interaction between a processing platform implementing the EDNN-based algorithm and other related equipment utilized in one or more of the use cases described above. For example, outputs generated by an EDNN-based algorithm can control one or more components of a related system. In some embodiments, the EDNN-based algorithm and the related equipment are implemented on the same processing platform, which may comprise a computer, a smartphone, a wearable device, a handheld sensor device or other type of processing device.

It should also be understood that the particular arrangements shown and described in conjunction with FIGS. 1-7 are presented by way of illustrative example only, and numerous alternative embodiments are possible. The various embodiments disclosed herein should therefore not be construed as limiting in any way. Numerous alternative arrangements of EDNN-based algorithms can be utilized in other embodiments. Those skilled in the art will also recognize that alternative processing operations and associated system entity configurations can be used in other embodiments.

It is therefore possible that other embodiments may include additional or alternative system elements, relative to the entities of the illustrative embodiments. Accordingly, the particular system configurations and associated algorithm implementations can be varied in other embodiments.

A given processing device or other component of an information processing system as described herein is illustratively configured utilizing a corresponding processing device comprising a processor coupled to a memory. The processor executes software program code stored in the memory in order to control the performance of processing operations and other functionality. The processing device also comprises a network interface that supports communication over one or more networks.

The processor may comprise, for example, a microprocessor, an ASIC, an FPGA, a CPU, a TPU, a GPU, an ALU, a DSP, or other similar processing device component, as well as other types and arrangements of processing circuitry, in any combination. For example, at least a portion of the functionality of at least one EDNN or an associated EDNN-based prediction and/or remediation algorithm provided by one or more processing devices as disclosed herein can be implemented using such circuitry.

The memory stores software program code for execution by the processor in implementing portions of the functionality of the processing device. A given such memory that stores such program code for execution by a corresponding processor is an example of what is more generally referred to herein as a processor-readable storage medium having program code embodied therein, and may comprise, for example, electronic memory such as SRAM, DRAM or other types of random access memory, ROM, flash memory, magnetic memory, optical memory, or other types of storage devices in any combination.

As mentioned previously, articles of manufacture comprising such processor-readable storage media are considered embodiments of the invention. The term “article of manufacture” as used herein should be understood to exclude transitory, propagating signals. Other types of computer program products comprising processor-readable storage media can be implemented in other embodiments.

In addition, embodiments of the invention may be implemented in the form of integrated circuits comprising processing circuitry configured to implement processing operations associated with implementation of an EDNN-based algorithm.

An information processing system as disclosed herein may be implemented using one or more processing platforms, or portions thereof.

For example, one illustrative embodiment of a processing platform that may be used to implement at least a portion of an information processing system comprises cloud infrastructure including virtual machines implemented using a hypervisor that runs on physical infrastructure. Such virtual machines may comprise respective processing devices that communicate with one another over one or more networks.

The cloud infrastructure in such an embodiment may further comprise one or more sets of applications running on respective ones of the virtual machines under the control of the hypervisor. It is also possible to use multiple hypervisors each providing a set of virtual machines using at least one underlying physical machine. Different sets of virtual machines provided by one or more hypervisors may be utilized in configuring multiple instances of various components of the information processing system.

Another illustrative embodiment of a processing platform that may be used to implement at least a portion of an information processing system as disclosed herein comprises a plurality of processing devices which communicate with one another over at least one network. Each processing device of the processing platform is assumed to comprise a processor coupled to a memory. A given such network can illustratively include, for example, a global computer network such as the Internet, a WAN, a LAN, a satellite network, a telephone or cable network, a cellular network such as a 3G, 4G or 5G network, a wireless network implemented using a wireless protocol such as Bluetooth, WiFi or WiMAX, or various portions or combinations of these and other types of communication networks.

Again, these particular processing platforms are presented by way of example only, and an information processing system may include additional or alternative processing platforms, as well as numerous distinct processing platforms in any combination, with each such platform comprising one or more computers, servers, storage devices or other processing devices.

A given processing platform implementing an EDNN-based algorithm as disclosed herein can alternatively comprise a single processing device, such as a computer, a smartphone, a wearable device, a handheld sensor device, or another type of processing device, that implements not only the EDNN-based algorithm but also at least one data source and one or more controlled components. It is also possible in some embodiments that one or more such system elements can run on or be otherwise supported by cloud infrastructure or other types of virtualization infrastructure.

It should therefore be understood that in other embodiments different arrangements of additional or alternative elements may be used. At least a subset of these elements may be collectively implemented on a common processing platform, or each such element may be implemented on a separate processing platform.

Also, numerous other arrangements of computers, servers, storage devices or other components are possible in an information processing system. Such components can communicate with other elements of the information processing system over any type of network or other communication media.

As indicated previously, components of the system as disclosed herein can be implemented at least in part in the form of one or more software programs stored in memory and executed by a processor of a processing device. For example, certain functionality disclosed herein can be implemented at least in part in the form of software.

The particular configurations of information processing systems described herein are exemplary only, and a given such system in other embodiments may include other elements in addition to or in place of those specifically shown, including one or more elements of a type commonly found in a conventional implementation of such a system.

For example, in some embodiments, an information processing system may be configured to utilize the disclosed techniques to provide additional or alternative functionality in other contexts.

It should again be emphasized that the embodiments of the invention as described herein are intended to be illustrative only. Other embodiments of the invention can be implemented utilizing a wide variety of different types and arrangements of information processing systems, networks and processing devices than those utilized in the particular illustrative embodiments described herein, and in numerous alternative processing contexts. In addition, the particular assumptions made herein in the context of describing certain embodiments need not apply in other embodiments. These and numerous other alternative embodiments will be readily apparent to those skilled in the art.

What is claimed is:

1. A method comprising:
obtaining data characterizing a given subject over time;
applying at least a portion of the obtained data to an encoder-decoder neural network adapted to generate a prediction of at least one change in at least one of behavior and physiology of the given subject from the obtained data; and
executing at least one automated remedial action relating to the given subject based at least in part on the generated prediction;
the encoder-decoder neural network being configured to learn one or more subject-specific anomaly thresholds based at least in part on reconstruction error of the encoder-decoder neural network;
wherein the method is performed by at least one processing device comprising a processor coupled to a memory.

2. The method of claim 1 wherein the encoder-decoder neural network is implemented at least in part utilizing a fully-connected neural network autoencoder architecture.

3. The method of claim 2 wherein the fully-connected neural network autoencoder comprises:
a hidden layer encoder;
a compressed layer; and
a hidden layer decoder;
wherein the hidden layer encoder receives an input data subsequence having a relatively high dimension and generates a first intermediate data subsequence having a relatively low dimension for delivery to the compressed layer; and
wherein the hidden layer decoder receives from the compressed layer a second intermediate data subsequence having the relatively low dimension and generates an output data sequence having the relatively high dimension and representing a reconstructed version of the input data subsequence having the relatively high dimension.

4. The method of claim 1 wherein the encoder-decoder neural network is implemented at least in part utilizing a gated recurrent unit (GRU) sequence-to-sequence architecture.

5. The method of claim 4 wherein the GRU sequence-to-sequence architecture comprises:
a GRU encoder comprising a first plurality of serially-connected GRU cells and having a specified hidden unit size; and
a GRU decoder comprising a second plurality of serially-connected GRU cells;
wherein a first one of the first plurality of serially-connected GRU cells of the GRU encoder receives an input data subsequence;
wherein a first one of the second plurality of serially-connected GRU cells of the GRU decoder receives one or more encoder outputs generated by a final one of the first plurality of serially-connected GRU cells of the GRU encoder; and
wherein a final one of the second plurality of serially-connected GRU cells of the GRU decoder generates a reconstructed version of the input data subsequence.

6. The method of claim 5 wherein the GRU encoder comprises a bidirectional GRU encoder and the first plurality of serially-connected GRU cells comprise respective bidirectional GRU cells, and further wherein the GRU decoder comprises a unidirectional GRU decoder and the second plurality of serially-connected GRU cells comprise respective unidirectional GRU cells.

7. The method of claim 1 wherein generating a prediction of at least one change in at least one of behavior and physiology of the given subject from the obtained data comprises detecting an anomaly based at least in part on reconstructed data generated by the encoder-decoder neural network from corresponding input data.

8. The method of claim 7 wherein detecting the anomaly comprises:
computing reconstruction error between the reconstructed data and the input data;
comparing the reconstruction error to a particular one of the one or more subject-specific anomaly thresholds; and
detecting the anomaly responsive to the reconstruction error exceeding the subject-specific anomaly threshold.

9. The method of claim 8 wherein the particular subject-specific anomaly threshold is determined based at least in part on a ratio of a true positive rate and a false positive rate for anomaly detection by the encoder-decoder neural network.

10. The method of claim 8 wherein comparing the reconstruction error to a particular one of the one or more subject-specific anomaly thresholds comprises:
generating anomaly scores for respective error vectors characterizing the reconstruction error between the reconstructed data and the input data for respective first time intervals, the anomaly scores being based at least in part on a specified distance metric;
processing the generated anomaly scores to generate a composite anomaly score for a second time interval having a duration longer than the first time interval; and
comparing the composite anomaly score to the particular subject-specific anomaly threshold.

11. The method of claim 1 wherein obtaining data characterizing the given subject over time further comprising obtaining data from at least one of:
one or more wearable devices of the given subject;
a smartphone of the given subject; and
one or more sensors associated with the given subject.

12. The method of claim 1 wherein learning of the encoder-decoder neural network is performed across multiple distinct features comprising one or more of:
at least one social behavior measure;
at least one sleep measure; and
at least one activity measure.

13. The method of claim 1 wherein executing at least one automated remedial action relating to the given subject based at least in part on the generated prediction comprises at least one of:
generating at least one control signal for controlling at least one controlled system component over a network;
generating at least a portion of at least one output display for presentation on at least one user terminal;
generating an alert for delivery to at least user terminal over a network; and
generating at least one output signal in a telemedicine application, wherein said at least one output signal in a telemedicine application comprises at least one of:
a prediction visualization signal for presentation on a user terminal;
diagnosis information transmitted over a network to a medical professional; and
prescription information transmitted over a network to a prescription-filling entity.

14. The method of claim 1 wherein at least a portion of the encoder-decoder neural network is implemented in at least one neural network integrated circuit.

15. A system comprising:
at least one processing device comprising a processor coupled to a memory;
the processing device being configured:
to obtain data characterizing a given subject over time;
to apply at least a portion of the obtained data to an encoder-decoder neural network adapted to generate a prediction of at least one change in at least one of behavior and physiology of the given subject from the obtained data; and
to execute at least one automated remedial action relating to the given subject based at least in part on the generated prediction;
the encoder-decoder neural network being configured to learn one or more subject-specific anomaly thresholds based at least in part on reconstruction error of the encoder-decoder neural network.

16. The system of claim 15 wherein generating a prediction of at least one change in at least one of behavior and physiology of the given subject from the obtained data comprises detecting an anomaly based at least in part on reconstructed data generated by the encoder-decoder neural network from corresponding input data.

17. The system of claim 16 wherein detecting the anomaly comprises:
computing reconstruction error between the reconstructed data and the input data;
comparing the reconstruction error to a particular one of the one or more subject-specific anomaly thresholds; and
detecting the anomaly responsive to the reconstruction error exceeding the subject-specific anomaly threshold.

18. A computer program product comprising a non-transitory processor-readable storage medium having stored therein program code of one or more software programs, wherein the program code, when executed by at least one processing device comprising a processor coupled to a memory, causes the processing device:
to obtain data characterizing a given subject over time;
to apply at least a portion of the obtained data to an encoder-decoder neural network adapted to generate a prediction of at least one change in at least one of behavior and physiology of the given subject from the obtained data; and
to execute at least one automated remedial action relating to the given subject based at least in part on the generated prediction;
the encoder-decoder neural network being configured to learn one or more subject-specific anomaly thresholds based at least in part on reconstruction error of the encoder-decoder neural network.

19. The computer program product of claim 18 wherein generating a prediction of at least one change in at least one of behavior and physiology of the given subject from the obtained data comprises detecting an anomaly based at least in part on reconstructed data generated by the encoder-decoder neural network from corresponding input data.

20. The computer program product of claim 19 wherein detecting the anomaly comprises:
computing reconstruction error between the reconstructed data and the input data;
comparing the reconstruction error to a particular one of the one or more subject-specific anomaly thresholds; and
detecting the anomaly responsive to the reconstruction error exceeding the subject-specific anomaly threshold.
